(19) United States
(12) Patent Application Publication
(10) Pub. No.: US 2026/0306417 A1
(43) Pub. Date: Oct. 8, 2026

(54) ORAL TARGETED NANO DELIVERY SYSTEMS CONTAINING GENIPOSIDE AND PREPARATION METHODS THEREOF

(71) Applicant: INSTITUTE OF CHINESE MATERIA MEDICA, CHINA ACADEMY OF CHINESE MEDICAL SCIENCES, Beijing (CN)

(72) Inventors: Aihua LIANG, Beijing (CN); Baoxin ZHENG, Beijing (CN); Chenyue LIU, Beijing (CN)

(73) Assignee: INSTITUTE OF CHINESE MATERIA MEDICA, CHINA ACADEMY OF CHINESE MEDICAL SCIENCES, Beijing (CN)

(21) Appl. No.: 19/292,860

(22) Filed: Aug. 6, 2025

(30) Foreign Application Priority Data
Apr. 3, 2025 (CN) 202510418881.7

(51) Int. Cl. A61K 9/51; A61K 9/00; A61K 31/7048

(52) U.S. Cl. CPC A61K 9/5115; A61K 9/0053; A61K 9/5161; A61K 9/5192; A61K 31/7048

(57) ABSTRACT

The present disclosure relates to oral targeted nano delivery systems containing geniposide, and preparation methods, and application thereof, and in particular, to the field of pharmaceuticals. The methods include synthesizing MSNs with a Stöber method using a template; removing the template, modifying a surface of the MSNs with amino groups to obtain MSN@NH₂; adding HA activated by NHS/EDCI to a suspension of the MSN@NH₂, and reacting to obtain MSN-HA; utilizing the MSN-HA, a chitosan solution, and sodium alginate to prepare Chi/Alg@MSN-HA nanoparticles (NPs); and loading the geniposide with the Chi/Alg@MSN-HA NPs in a proportional ratio to prepare geniposide-loaded Chi/Alg-GE@MSN-HA NPs. In the present disclosure, by constructing a targeted nano delivery system and changing the site of administration, the anti-UC efficacy of the geniposide is improved and a solution is provided for the safety problem of the geniposide by realizing the localized release of the geniposide in the colon.

### CROSS-REFERENCE TO RELATED APPLICATIONS

This application claims priority to the Chinese Patent Application No. 202510418881.7, filed on Apr. 3, 2025, the entire contents of which are incorporated herein by reference.

### TECHNICAL FIELD

The present disclosure relates to the field of pharmaceuticals, and in particular, to targeted nano delivery systems containing geniposide configured for oral administration, methods of preparation, and applications thereof.

### BACKGROUND

Ulcerative colitis (UC) is an inflammatory bowel disease with an insidious disease location, characterized by diarrhea, blood in the stool, and intestinal damage, and the clinical Chinese medicine dialectic of the UC is predominantly of the type of damp-heat-impregnated intestinal evidence. The Chinese medicinal herb is the dried ripe fruit of Gardenia jasminoides Ellis, a plant belonging to the Rubiaceae family. As recorded in Shennong Bencaojing, the gardenia tastes bitter, has a cold nature, enters the heart, the lungs, and the triple jiao meridian, possesses the effects of purging fire to relieve restlessness, clearing heat and draining dampness, cooling blood and detoxifying, and used for damp-heat jaundice, fire-toxin sores, and other conditions. Since the geniposide (GE) is the main active ingredient of the gardenia to exert anti-inflammatory and other pharmacodynamic effects, many scholars have carried out some basic research on the pharmacodynamic activity, toxicity, and toxicology of the GE, which has proved the potential application value of the GE in the UC. However, the rapid degradation of the GE after oral administration results in a lower content of the pharmaceutical component reaching the colon, which affects the efficacy, and an increased dosage may lead to hepatotoxicity. In their study on the comparison of toxicity of geniposide administered via different routes, Wang Enli et al. pointed out that in the gavage and injection groups, the hepatotoxicity induced by gavage is more significant, which may be related to the decomposition of the GE into genipin in the gastrointestinal tract. However, for the treatment of colitis via oral administration, a relatively large dosage is required, and oral administration will significantly increase the hepatotoxicity of geniposide. Feng Xiaoyi et al. pointed out in their study that high-dose administration of geniposide can cause pathological damage to the kidneys. Currently, geniposide is mostly administered via enema for the treatment of colitis; however, enema administration is inconvenient. Therefore, how to administer geniposide orally for the treatment of colitis without affecting its efficacy remains an urgent problem to be solved.

Chinese Patent Publication No. 107865822 A discloses methods for preparing a hydrogel drug slow-release carrier material doped with mesoporous silica nanoparticles (MSNs) and applications thereof, and mentions that using gelatin, sodium alginate (SA), and the MSNs as raw materials, Gelatin-SA semi-interpenetrating composite gel containing MSNs is firstly prepared by physical cross-linking of Ca²⁺, and then the Gelatin-SA semi-interpenetrating composite gel containing MSNs is modified with polyelectrolyte membrane by chitosan (CS), thus obtaining stabilized Gelatin-SA-CS (MSNs) composite hydrogel. The slow-release effect of the Gelatin-SA-CS (MSNs) composite hydrogel on pharmaceuticals was investigated using bovine serum albumin (BSA), metformin hydrochloride (MH), and indomethacin (IDM) as model pharmaceuticals. However, it is known from the carrier pharmaceutical in the patent that the carrier in the patent is mainly enriched in the intestinal tract and can't target the colon to realize colon targeted drug delivery.

The Chinese Patent Publication No. CN111265670 A discloses galactoglycated chitosan-coated mesoporous silica carriers and applications thereof, and mentions that based on the mesoporous silica carriers and combined with galactoglycated chitosan (GC), 5-fluorouracil (5-FU) in combination with leucovorin calcium (LV) for targeted treatment of colon cancer is realized. Although the carrier in the patent also targets the colon region, but specifically targets tumor cells in the colon, and the carrier cannot target CD44 macrophages in the inflammatory sites of the colon.

The Chinese Patent Publication No. CN 118806914 A discloses drug delivery systems, preparation methods, and applications thereof, and mentions the drug delivery system including a mucin, a genipin, an oil phase, and an emulsifier, and the preparation method of the drug delivery system including: (1) mixing the oil phase and the emulsifier to obtain a mixture 1; (2) mixing an aqueous solution containing the mucin and the mixture 1 for emulsification to obtain a mixture 2; (3) mixing the mixture 2 and the solution containing the genipin for crosslinking to obtain a mixture containing the drug delivery system. The genipin is a metabolite of the GE and is used as a part of the drug delivery system. But geniposide cannot crosslink with the drug delivery system, making it impossible to target the colonic for therapeutic efficacy. Therefore, a new colonic drug delivery system capable of targeting for loading the GE drug is needed.

### SUMMARY

The present disclosure provides oral targeted nano delivery systems containing geniposide, methods for preparation, and application thereof to solve the problems in the prior art. The oral colon-targeted delivery system in the present disclosure alternative to enemas is intended to improve compliance and targeting.

A first aspect of the present disclosure provides a method for preparing an oral targeted nano delivery system containing geniposide. The method includes:

### Step (a): Preparation of a Targeted Nano Delivery System

synthesizing mesoporous silica nanoparticles (MSNs) with a Stöber method using a template; removing the template, modifying a surface of the MSNs with amino groups to obtain MSN@NH₂; adding hyaluronic acid (HA) activated by N-hydroxysuccinimide (NHS)/1-ethyl-3-(3-dimethylaminopropyl) carbodiimide hydrochloride (EDCI) to a suspension of the MSN@NH₂, and reacting to obtain MSN-HA; utilizing the MSN-HA, a chitosan solution, and sodium alginate to prepare Chi/Alg@MSN-HA nanoparticles (NPs).

### Step (b): Preparation of Geniposide-Loaded Chi/Alg-GE@MSN-HA NPs

loading the geniposide with the Chi/Alg@MSN-HA NPs in a proportional ratio to prepare the geniposide-loaded Chi/Alg-GE@MSN-HA NPs.

A second aspect of the present disclosure provides an oral targeted nano delivery system containing geniposide. The oral targeted nano delivery system containing geniposide is prepared by the method described above.

A third aspect of the present disclosure provides a method of treating ulcerative colitis, comprising administering to a patient suffering from ulcerative colitis an effective amount of geniposide-loaded Chi/Alg-GE@MSN-HA NPs.

### BRIEF DESCRIPTION OF THE DRAWINGS

The application file contains at least one drawing executed in color. Copies of this patent application publication with color drawing(s) will be provided by the Office upon request and payment of the necessary fee.

In order to more clearly illustrate the embodiments in the present disclosure or technical solutions in the prior art, the drawings required to be used in the description of the embodiments, or the prior art, are briefly described below. Obviously, the drawings in the following description are merely exemplary, and other embodiments may be obtained by those of ordinary skill in the art, without creative labor, based on the drawings provided.

The structure, the proportional ratio, the size, etc., shown in the present disclosure are only used to match the content of the present disclosure for a person familiar with the technology to understand and read, not to limit the implementation of the present disclosure, and do not have technical significance. Any structural modification, change in the proportional ratio, or adjustment in the size, without affecting the efficacy achievable by or the objectives attainable through the present disclosure, should remain within the scope of the technical content disclosed in the present disclosure.

FIG. 1 shows transmission electron microscopy (TEM) characterization and particle size distribution statistics of MSNs at different reaction temperatures according to some embodiments of the present disclosure.

FIG. 2 shows TEM characterization and particle size distribution statistics of MSNs under different molar dosages of pore expanders according to some embodiments of the present disclosure.

FIG. 3 shows Brunauer-Emmet-Teller (BET) analysis of adsorption-desorption isotherms and pore diameter distribution profiles of a plurality of MSNs at different reaction temperatures and different dosages of pore expanders according to some embodiments of the present disclosure, where (A)—60° C., (B)—70° C., (C)—80° C., (D)—90° C., (E)—T1:E-TMB/MSNs (mol/mol)=1:1, (F)—T2:E-TMB/MSNs (mol/mol)=2:1, and (G)—T4:E-TMB/MSNs (mol/mol)=4:1.

FIG. 4 shows a scanning electron microscopy (SEM) image, particle size distribution, and Zeta potential of a nano delivery system according to some embodiments of the present disclosure, where (A)—an SEM image of nanocarriers Chi/Alg-MSN-HA, (B)—particle size distribution results of MSNs, MSN-HA, and Chi/Alg-MSN-HA, (C)—Zeta potential measurements of different intermediates during nanocarrier preparation, with the x-axis labels in (C) denoting: C for chitosan coating, A for sodium alginate coating, and subscripts indicating a count of self-assembled layers.

FIG. 5 is a schematic diagram of Chi/Alg-GE@MSN-HA NPs according to some embodiments of the present disclosure.

FIG. 6 shows Fourier transform infrared (FTIR) spectrum of MSNs, MSN@NH₂, MSN-HA, Chi/Alg-MSN-HA, Chi/Alg-GE@MSN-HA NPs, and free GE according to some embodiments of the present disclosure.

FIG. 7 shows drug loading efficiency and encapsulation efficiency test results (n=3) of Chi/Alg-GE@MSN-HA with different feeding ratios according to Embodiment 1 of the present disclosure.

FIG. 8 shows changes (n=6) in particle size and Zeta potential of Chi/Alg-GE@MSN-HA NPs sampled on different days during long-term frozen storage according to Embodiment 2 of the present disclosure.

FIG. 9 shows a concentration standard curve of geniposide solution according to Embodiment 2 of the present disclosure.

FIG. 10 shows HPLC chromatogram profiles according to Embodiment 2 of the present disclosure, where (A)—blank solvent, and (B)—geniposide standard solution.

FIG. 11 shows cumulative release profiles (n=6) of GE@MSN-HA NPs and Chi/Alg-GE@MSN-HANPs under a simulated gastrointestinal condition in vitro according to Embodiment 2 of the present disclosure.

FIG. 12 is a diagram illustrating cytotoxicity evaluation (n=6) of MSNs, MSN-HA NPs, and Chi/Alg-MSN-HA NPs according to Embodiment 2 of the present disclosure.

FIG. 13 shows confocal laser scanning microscopy (CLSM) analysis of uptake and distribution of LPS-induced RAW264.7 inflammation model after 30 min, 1 h, 2 h, and 4 h incubation with free coumarin 6 (C6), C6@MSN-HA NPs, and Chi/Alg-C6@MSN-HA NPs in vitro (n=6) according to Embodiment 2 of the present disclosure.

FIG. 14 shows results of colon-targeting verification of an oral nanocarrier system Chi/Alg-Dir@MSN-HA in mice according to Embodiment 2 of the present disclosure, where (A)—In vivo imaging photographs of the mice administered free Dir, Dir@MSN-HA, and Chi/Alg-Dir@MSN-HA by gavage in 0-24 h, and (B)—In vivo fluorescence intensity statistics at different time points (n=6), (compared to a free Dir group, *p<0.05 and ***p<0.001).

FIG. 15 shows in vitro imaging photographs of the colon isolated from mice after gavage administration of Dir, Dir@MSN-HA, and Chi/Alg-Dir@MSN-HA for 24 h according to Embodiment 2 of the present disclosure (n=3).

FIG. 16 shows in vivo efficacy evaluation of Chi/Alg-GE@MSN-HA according to Embodiment 2 of the present disclosure; where (A)—change curves in body weight of the mice during an administration observation period; (B)—Disease Activity Index (DAI) scores of the mice; (C)—statistics of colonic lengths of a plurality of groups of mice; and (D)—photographs of the colons (n=6); (compared to the control group, *p<0.05, **p<0.01, and ***p<0.001; compared to the model group, ^{#}p<0.05, ^{##}p<0.01, and ^{###}p<0.001).

FIG. 17 shows organ coefficient assessments of UC murine model of a plurality of groups of mice (n=6) after administration according to Embodiment 2 of the present disclosure.

FIG. 18 shows histopathological paraffin sections of mouse colons according to Embodiment 2 of the present disclosure; where, (A)—H&E staining; and (B)—histopathological scores (n=6) (compared to the control group, ***p<0.001; compared to the model group, ^{#}p<0.05, ^{##}p<0.01, and ^{###}p<0.001).

### DETAILED DESCRIPTION

The following particular and specific embodiments illustrate the manner of implementation of the present disclosure, and a person familiar with the art can readily understand other advantages and efficacies of the present disclosure by the contents disclosed herein. It is obvious that the described embodiments are a part of the present disclosure, and not all embodiments. Based on the embodiments of the present disclosure, other embodiments obtained by one in the art without creative labor are within the scope of protection of the present disclosure.

Some embodiments of the present disclosure provide a method for preparing an oral targeted nano delivery system containing geniposide. The method includes the following steps.

### Step (a): Preparation of a Targeted Nano Delivery System.

Synthesize MSNs with a Stöber method using a template; remove the template, modify a surface of the MSNs with amino groups to obtain MSN@NH₂; add hyaluronic acid (HA) activated by N-hydroxysuccinimide (NHS)/1-ethyl-3-(3-dimethylaminopropyl) carbodiimide hydrochloride (EDCI) to a suspension of the MSN@NH₂, and react to obtain MSN-HA; utilize the MSN-HA, a chitosan solution, and sodium alginate to prepare Chi/Alg@MSN-HA NPs.

### Step (b): Preparation of Geniposide-Loaded Chi/Alg-GE@MSN-HA NPs

Load geniposide with the Chi/Alg@MSN-HANPs in a proportional ratio to prepare the geniposide-loaded Chi/Alg-GE@MSN-HA NPs.

The geniposide-loaded Chi/Alg-GE@MSN-HA NPs refers to as the oral targeted nano delivery system containing geniposide. The above preparation process is efficient and simple, with a mild condition, and suitable for large-scale production. Silica is biologically inert and meets the FDA standard for pharmaceutical excipients, making silica suitable for in vivo application. An internal skeletal mesostructure of the MSNs endows the nanocarrier with an ultra-high specific surface area (954.37 m²/g), which significantly enhances the drug loading capacity. A layer of the hyaluronic acid and a layer of the sodium alginate/chitosan in a shell layer are both natural polysaccharides with good biocompatibility.

The Chi/Alg-GE@MSN-HA NPs prepared are of uniform particle size, with a high encapsulation efficiency and a high drug loading capacity, and can overcome the obstacles of the physiological environment of the upper gastrointestinal tract after oral administration, and are sequentially targeted to the colonic focus and the nearby macrophages step by step, which obviously improves the retention of a targeted colon site, fully improves anti-UC efficacy of the geniposide, provides a new strategy for improving the bioavailability of the geniposide and the precise treatment of the UC.

In some embodiments, in the step (a), synthesizing the MSNs with the Stöber method includes: using cetyltrimethylammonium bromide (CTAB) as the template, 1,3,5-trimethylbenzene (TMB) as a pore expander, and tetraethyl orthosilicate (TEOS) as a silicon source.

In some embodiments, in the step (a), synthesizing the MSNs with the Stöber method includes: using cetyltrimethylammonium chloride (CTAC) as the template, TMB as the pore expander, and TEOS as the silicon source.

In some embodiments, synthesizing the MSNs with the Stöber method includes the following steps. Dissolve the CTAB or the CTAC in deionized water to obtain a solution and add sodium hydroxide to the solution. When the CTAB or the CTAC is completely dissolved, an appropriate amount of the TMB is added quickly and stirred continuously. An appropriate amount of the TEOS is then added dropwise with a pressure-equalizing dropping funnel to obtain a synthesized material. Particles are collected from the synthesized material by centrifugation and washed with methanol. The synthesized material (CTAB@MSNs or CTAC@MSNs) is dispersed in a mixed hydrochloric acid/methanol solution and stirred overnight to remove the template. Particles are collected from the synthesized material by centrifugation. The particles are washed with methanol and dried. In some embodiments, a temperature of the stirred overnight is in a range of 55° C. to 65° C. In some embodiments, the temperature of the stirred overnight is in a range of 45° C. to 75° C. In some embodiments, a drying temperature is in a range of 65° C. to 90° C. In some embodiments, the drying temperature is in a range of 70° C. to 90° C. In some embodiments, the drying temperature is in a range of 75° C. to 85° C. In some embodiments, the drying temperature is 85° C.

In some embodiments, a molar ratio of TMB/MSNs is in a range of 1:2 to 4:1. In some embodiments, the molar ratio of TMB/MSNs is in a range of 1:1 to 4:1. In some embodiments, the molar ratio of TMB/MSNs is in a range of 1:1 to 2:1. In some embodiments, the molar ratio of TMB/MSNs is 2:1. The molar ratio of TMB/MSNs refers to a molar ratio of TMB to TEOS before reaction in the step (a). The molar mass of TEOS is 208.33 g/mol, and its density is 0.9356 g/cm³. The molar mass of TMB is 240.34 g/mol, and its density is 0.867 g/cm³. Merely as an example, The volume of TEOS is 3.75 mL, and its molar amount is calculated as: 3.75 mL×0.9356 g/cm³÷208.33 g/mol=0.0168 mol; The volume of TMB is 4.50 mL, and its molar amount is calculated as: 4.50 m¹×0.867 g/cm³÷240.34 g/mol=0.0162 mol. At this point, the molar ratio is approximately 1:1. In some embodiments, to optimize different molar ratios, the adding volume of TMB is varied sequentially.

In some embodiments, in the step (a), modifying the surface of the MSNs with the amino groups includes: dispersing the MSNs in methanol, adding 3-aminopropyltriethoxysilane (APTES), reacting at room temperature overnight, centrifuging an obtained product, washing with methanol, and drying to obtain the MSN@NH₂.

In some embodiments, a ratio of the MSNs:the methanol:the APTES=0.8-1.2 g:0.8-1.2 mL:0.8-1.2 μL.

In some embodiments, the ratio of the MSNs:the methanol:the APTES is 1 g:1 mL:1 μL.

In some embodiments, the step (a) includes dispersing the MSN@NH₂ in a 4-morpholineethanesulfonic acid (MES) buffer solution to obtain the suspension of MSN@NH₂.

In some embodiments, the MSN@NH₂ is added to the MES buffer solution and stirred at room temperature to obtain a solution A. An appropriate amount of the hyaluronic acid is added to the MES buffer solution, stirred at room temperature until fully dissolved, an appropriate amount of the EDCI and the NHS are added, and carboxyl groups of the hyaluronic acid are activated by stirring at room temperature to obtain a solution B. The solution A and the solution B are mixed at a volume ratio of 1:1, stirred at room temperature for 24 h, washed with deionized water, and freeze-dried in a lyophilizer. In some embodiments, a mass ratio of the MSN@NH₂ to the hyaluronic acid is in a range of 3:1 to 3:2. In some embodiments, the mass ratio of the MSN@NH₂ to the hyaluronic acid is in a range of 5:2 to 5:4. In some embodiments, the mass ratio of the MSN@NH₂ to the hyaluronic acid is 5:3.

In some embodiments, in the step (a), the preparation of the Chi/Alg@MSN-HA NPs includes dispersing the MSN-HA in the chitosan solution, stirring at room temperature, suspending a resulting product in a sodium alginate solution, stirring to react, and centrifuging to obtain the Chi/Alg@MSN-HA NPs.

In some embodiments, the chitosan solution is a phosphate buffered saline (PBS) buffer solution.

In some embodiments, in the step (b), the preparation of the geniposide-loaded Chi/Alg-GE@MSN-HA NPs includes dispersing the geniposide and the Chi/Alg@MSN-HA NPs in anhydrous ethanol in the proportional ratio, subjecting to ultrasonication, stirring at room temperature overnight, centrifuging, washing repeatedly with deionized water and anhydrous ethanol, and freeze-drying to obtain the geniposide-loaded Chi/Alg-GE@MSN-HANPs. In some embodiments, in the step (b), a mass ratio of the geniposide to the Chi/Alg@MSN-HA NPs is in a range of 1:10 to 10:1. In some embodiments, the mass ratio of the geniposide to the Chi/Alg@MSN-HA NPs is in a range of 1:10 to 2:1. In some embodiments, the mass ratio of the geniposide to the Chi/Alg@MSN-HA NPs is in a range of 1:2 to 2:1. In some embodiments, the mass ratio of the geniposide to the Chi/Alg@MSN-HA NPs is in a range of 1:2 to 2:1. In some embodiments, the mass ratio of the geniposide to the Chi/Alg@MSN-HA NPs is 1:1.

Some embodiments of the present disclosure provide a method of treating ulcerative colitis. The method of treating ulcerative colitis includes administering to a patient suffering from ulcerative colitis an effective amount of geniposide-loaded Chi/Alg-GE@MSN-HA NPs.

### 1. Optimization Results and Analysis of Materials

The MSNs are high-quality skeletal materials for drug delivery. The drug loading property and the in vivo drug releasing process of the MSNs are closely related to the structural property of the MSNs. The structural property of the MSNs includes a structural arrangement regularity of a material skeleton, a particle size, a polydispersity index (PDI), a Zeta potential, a pore diameter distribution, a specific surface area, a pore volume, etc. A reaction temperature and a dosage of the pore expander are key factors affecting the structural property of the MSNs. The PDI is an index of a degree of homogeneity of the particle size. The more homogeneous the particle size, the more stable the structural property of the MSNs. The Zeta potential is an important indicator of surface charges of nanoparticles, which has a large impact on dispersion and later modification. The greater a negative value of the Zeta potential of the MSNs, the more stable the dispersion of the MSNs in a solution, and the more favorable for later modification.

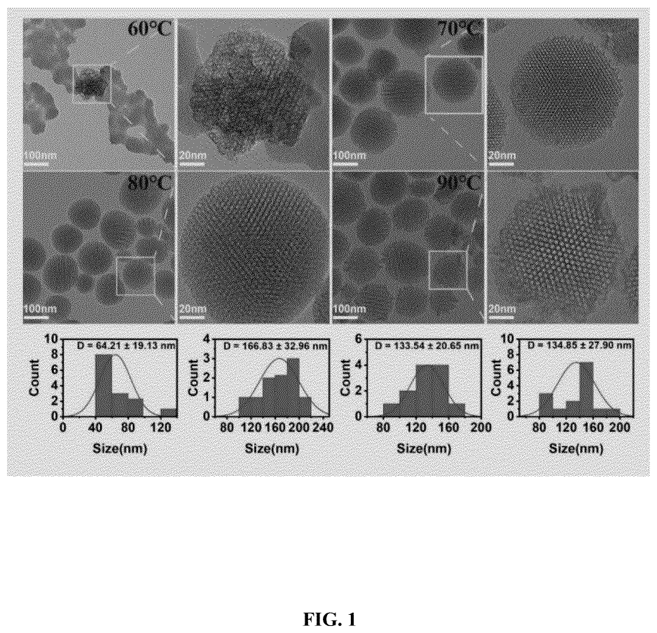

A preparation process of the MSNs is optimized by taking the reaction temperature and the dosage of the pore expander as conditional variables, and morphology and the particle size observed by TEM and the Zeta potential as indexes. FIG. 1 shows transmission electron microscopy (TEM) characterization and particle size distribution statistics of MSNs at different reaction temperatures according to some embodiments of the present disclosure. As shown in FIG. 1, the results of the TEM show that the skeleton of the MSNs is fuzzy when the reaction temperature is 60° C. The crystal growth is still in a primary stage, and a regular mesoporous arrangement and a framework structure have not yet been formed, which is not able to meet requirements of the preparation. With the increase of the reaction temperature, the particle size of the MSNs gradually increases. When the reaction temperature is in a range of 70° C. to 90° C., the mesoporous arrangement of the MSNs is gradually clearer, and the size of the mesopores increases significantly. The particle size in the TEM shows that when the reaction temperature is 60° C., 70° C., 80° C., and 90° C., an average particle size is 64.21±19.13 nm, 166.83±32.96 nm, 133.54±20.65 nm, and 134.85±27.90 nm, respectively. When the reaction temperature is 70° C., despite a regular mesoporous arrangement in an internal framework and a better roundness, the presence of spiny edges may be unfavorable for the later modification. When the reaction temperature is 90° C., mesopores on the surface are obviously enlarged and uniformly arranged, but the structure of the outer layer appears to be partially collapsed. It is possible that a higher temperature exacerbates Brownian motion and collisions of reacting molecules and thus may be unfavorable for the end stage of surface growth of a mesoporous material. Contrarily, when the reaction temperature is 80° C., the mesopores are uniformly arranged, and the end stage of the surface growth of the mesoporous material is more intact without localized structural collapse. As shown in Table 1, results of a Malvern particle size, a Zeta potential, and a PDI value show that nanoparticles obtained at the reaction temperature of 80° C. have a particle size of 120.34 nm, which is less than 200 nm, and favorable for overcoming drug delivery barrier. The Zeta potential on the surface is-35.39 mV, an absolute value is more than 30 mV, which is favorable for dispersion and stability in a solution system. The PDI is 0.18, which is less than 0.20, indicating that uniformity of the particle size is good, which is favorable for achieving more stable drug loading and drug release. Therefore, based on the analytical morphology and the structure of the TEM characterization results of the MSNs and the results of the Malvern particle size, the Zeta potential, and the PDI, an optimal reaction temperature of the MSNs is 80° C.

TABLE 1

| Reaction | | | |
|---|---|---|---|
| temperature/° C. | Particle size/nm | PDI | Zeta potential/mV |
| 60 | 70.54 ± 1.75 | 0.23 ± 0.03 | −10.39 ± 0.24 |
| 70 | 112.34 ± 1.78 | 0.28 ± 0.02 | −20.01 ± 0.12 |
| 80 | 120.34 ± 1.90 | 0.18 ± 0.02 | −35.39 ± 0.48 |
| 90 | 160.34 ± 2.20 | 0.62 ± 0.05 | −29.39 ± 0.19 |

The dosage of the pore expander TMB, on the one hand, may change the particle size of the MSNs and thus affect the drug loading and the drug release, on the other hand, may affect the structural properties of the MSNs. Results of different molar ratios of TMB/MSNs are shown in Table 2.

TABLE 2

| TMB/MSNs/ | Particle | | Zeta |
|---|---|---|---|
| mol/mol | size/nm | PDI | potential/mv |
| 1:2 | 90.54 ± 1.05 | 0.23 ± 0.03 | −24.39 ± 0.24 |
| 1:1 | 162.34 ± 1.33 | 0.17 ± 0.03 | −30.11 ± 0.29 |
| 2:1 | 125.17 ± 1.18 | 0.15 ± 0.02 | −38.17 ± 0.61 |
| 4:1 | 120.34 ± 2.12 | 0.61 ± 0.09 | −28.39 ± 0.19 |

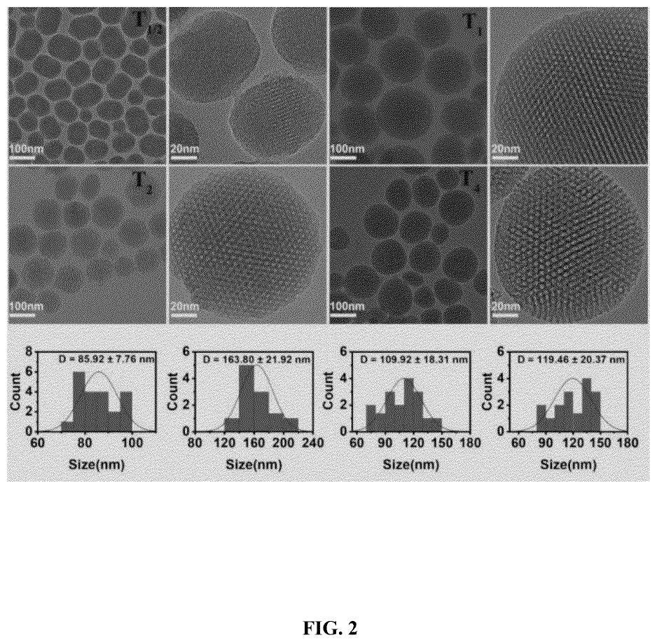

FIG. 2 shows TEM characterization and particle size distribution statistics of MSNs under different molar dosages of pore expanders according to some embodiments of the present disclosure. Results of the TEM show that mesoporous channels of the MSNs are blurred and have a shape between a long columnar and an ellipse with a ratio of a length of a long axis of the mesoporous channels to a length of a short axis of the mesoporous channels of about 2:1 when the molar ratio of TMB/MSNs (mol/mol) is 1:2. As the dosage of the pore expanders continues to increase, a roundness gradually improves, which indicates that the dosage of the pore expanders, in addition to affecting the particle size of the MSNs, also affect a shape of the MSNs, thereby enriching the preparation needs under different drug delivery functions. When the molar ratio of TMB/MSNs (mol/mol) is 1:1, 2:1, and 4:1, the mesoporous arrangement is regular and well-rounded, and average particle sizes under TEM are 163.80±21.92 nm, 109.92±18.31 nm, and 119.46±20.37 nm, respectively. The results of the TEM show that an order of homogeneity of the particles may be judged as T4<T1/2<T1<T2 (FIG. 2). Meanwhile, results of the Malvern particle size, the Zeta potential and the PDI value show that the PDI value is less than 0.20 when TMB/MSNs (mol/mol) is 1:1 and 2:1. When TMB/MSNs (mol/mol) is 1:2, the particle size is even smaller, 90.54±1.05 nm, which is similar to the results of the TEM. After treatment with pore expanders, the absolute value of the Zeta potential of the MSNs increases, indicating more uniform surface charge distribution and higher charge density on the MSNs after pore expanding, which is favorable for stabilization of dispersion in a solvent system. When TMB/MSNs (mol/mol) is 1:1 and 2:1, the absolute value of the Zeta potential is >30 mV, especially when the molar ratio of TMB/MSNs is 1:2, the Zeta potential is −38.17±0.61 mV (as shown in the Table 2). Therefore, an optimal condition for the dosage of the pore expanders is 2:1 for TMB/MSNs (mol/mol).

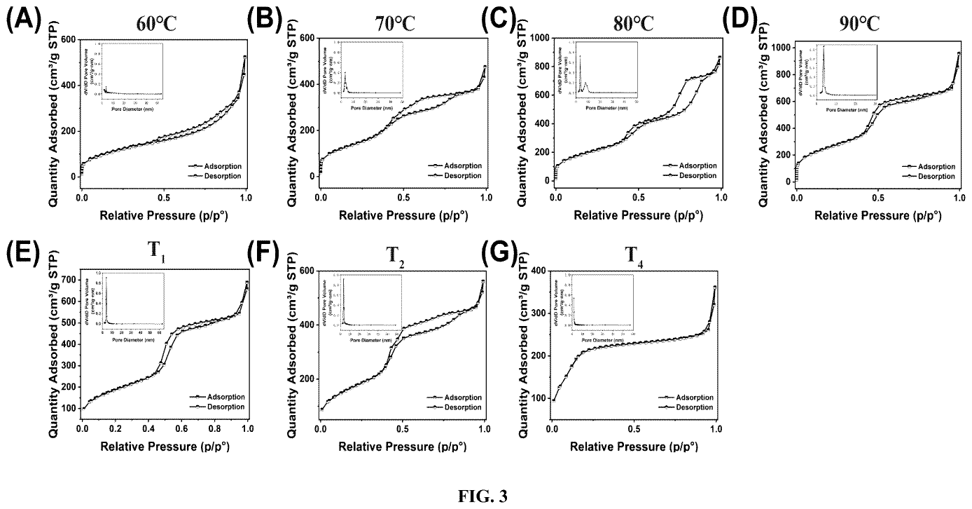

According to the types of MSNs reported so far, the types of MSNs mainly include MCM series (e.g., MCM-41 and MCM-48) and SBA series (e.g., SBA-15 and SBA-16). The MSMs synthesized with the Stöber method in the present disclosure are different from the above types of MSNs in that the internal pore diameter distribution and the shape of the mesoporous channels are short-range ordered rather than long-range ordered. Therefore, the potential of the drug loading of the MSNs is further investigated after an optimization of conditions, i.e., 2:1 for TMB/MSNs (mol/mol) and the reaction temperature of 80° C. In this experiment, Brunauer-Emmet-Teller (BET) technique is used to determine the specific surface area, a pore volume, the average pore diameter, and other indexes of the MSNs under different pre-stage conditions, and adsorption-desorption isotherms are plotted to determine the effect on the properties of the drug loading. Due to an irregular shape between the long columnar and the ellipse of the MSNs under the condition of TMB/MSNs (mol/mol) of 1:2, the MSNs are not suitable for developing as an application of the drug loading in a later stage. Therefore, the BET test is only performed on the MSNs under other conditions. A-G in FIG. 3 show BET adsorption-desorption isotherms and pore diameter distribution profiles of MSNs under different conditions in turn.

FIG. 3 shows BET analysis of adsorption-desorption isotherms and pore diameter distribution profiles of a plurality of MSNs at different reaction temperatures and different dosages of pore expanders according to some embodiments of the present disclosure, where (A)—60° C., (B)—70° C., (C)—80° C., (D)—90° C., (E)—T1:E-TMB/MSNs (mol/mol)=1:1, (F)—T2:E-TMB/MSNs (mol/mol)=2:1, and (G)—T4:E-TMB/MSNs (mol/mol)=4:1. Results of the BET show that the adsorption-desorption isotherms and pore diameter distribution profiles at the reaction temperature of 60° C. are characterized by low relative pressure hysteresis, steeper curves, and a narrow hysteresis loop, which belong to an H4 type hysteresis loop. The results of the BET indicate that the MSNs have a mixed structure with mesopores and micropores, with steeper curves, no obvious plateau area, and a relatively narrow hysteresis loop, which reflects a narrow and inhomogeneous pore channel structure of the MSNs, which is consistent with the results of TEM. The hysteresis loop of FIG. 3C belongs to an H2 type, which indicates that the MSNs have an ink-bottle-like mesoporous structure with a large internal space, thereby providing a large number of storage sites for drug molecules, which can accommodate more drug molecules, resulting in higher drug loading, which helps to improve therapeutic efficacy of drug delivery system and reduce a count of drug administrations. In addition, materials with a mesoporous structure in general tend to be more biocompatible. Meanwhile, the MSNs in FIG. 3C have a stable pore structure, and the stable pore structure does not decompose or release a large amount of the drug rapidly in vivo, but rather acts in a slow-release manner, which further improves safety and reliability of the drug loading system. The hysteresis loop of FIG. 3F belongs to an H3 type, indicating that the MSNs possess slit-like mesopores, and usually have a potential of high drug loading, good slow-release performance, and drug stability. A specific surface area, a pore volume, and an average pore diameter of a plurality of the MSNs at different reaction temperatures and different dosages of the pore expanders are analyzed by BET. The results at different reaction temperatures show that the specific surface area increases and then decreases as the reaction temperature increases. When the reaction temperature is 80° C., the SBET (m²/g) reaches a maximum of 745.98 m²/g, the pore volume also reaches a maximum of 1.07 m³/g, and the pore diameter is 5.98 nm. The results of different dosages of the pore expander show that the specific surface area first increases and then decreases as the dosage of TMB increases. A maximum value of the specific surface area reaches 954.37 m²/g, the pore volume reaches 1.16 m³/g, and the pore diameter is 4.13 nm when the reaction temperature is 80° C. and TMB/MSNs (mol/mol) is 1:2, which indicates that the condition is the most favorable for drug loading. With a larger specific surface area, an appropriate pore volume, and an appropriate pore diameter, usually mesopores with a pore diameter of 2-7 nm, possess better drug loading characteristics (as shown in Table 3).

TABLE 3

| Reaction | TMB/MSNs/ | | | |
|---|---|---|---|---|
| temperature/° C. | mol/mol | S_{BET}/m²/g | Vₜ/cm³/g | W_{BJH}/nm |
| 60 | / | 420.94 | 0.52 | 7.79 |
| 70 | / | 518.05 | 0.58 | 4.66 |
| 80 | / | 745.98 | 1.07 | 5.98 |
| 90 | / | 679.67 | 0.84 | 5.34 |
| 80 | 1:1 | 709.26 | 0.40 | 3.77 |
| 80 | 2:1 | 954.37 | 1.16 | 4.13 |
| 80 | 4:1 | 631.64 | 0.72 | 4.47 |

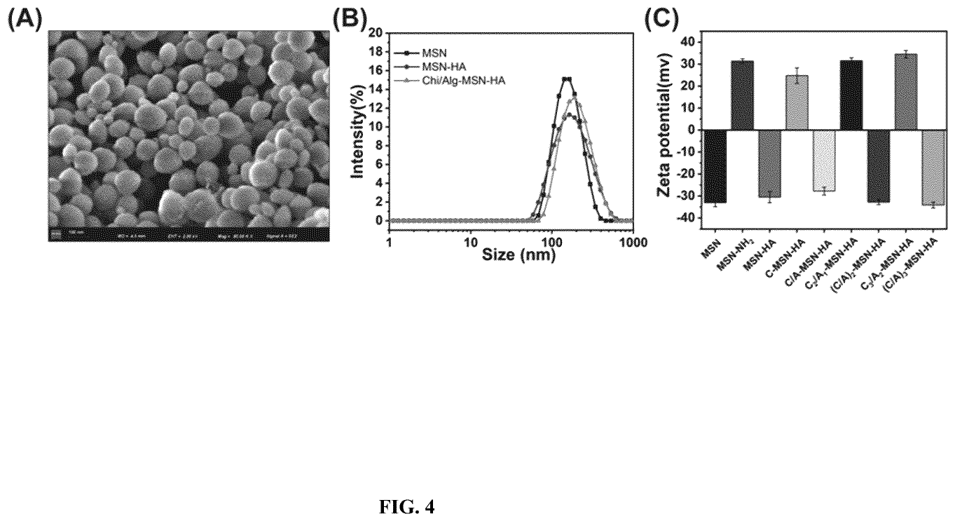

FIG. 4 shows a SEM image, particle size distribution, and Zeta potential of a nano delivery system according to some embodiments of the present disclosure, where (A)—an SEM image of nanocarriers Chi/Alg-MSN-HA, (B)—particle size distribution results of MSNs, MSN-HA, and Chi/Alg-MSN-HA, (C)—Zeta potential measurements of different intermediates during nanocarrier preparation, with the x-axis labels in (C) denoting: C for chitosan coating, A for sodium alginate coating, and subscripts indicating a count of self-assembled layers. Results of the SEM show that the nanocarriers Chi/Alg-MSN-HA are spherical nanoparticles with good monodispersity and no obvious agglomeration. The particle size of the nanocarriers Chi/Alg-MSN-HA is about 122.18 nm, a surface is uniformly distributed with dot-like bumps, and a size distribution is relatively homogeneous (FIG. 4A). The test results of the particle size show that the particle sizes of the MSNs, the MSN-HA, and the Chi/Alg-MSN-HA are 128.15 nm, 148.11 nm and 158.17 nm, respectively, indicating that a modification of a layer of hyaluronic acid and a layer of sodium alginate/chitosan on an outer layer of the MSNs is successful and has an increasing effect on the particle size, but the particle size still belongs to a range of smaller than 200 nm, which still retains a better nano-size effect, is favorable for overcoming biological barriers to drug delivery (FIG. 4B). The test results of the Zeta potential show that an initial surface Zeta potential of the MSNs is about −33.03 mV and is about 31.4 mV after surface animation. A Zeta potential of hyaluronic acid-functionalized MSN-HA is about −30.5 mV. To prevent degradation of nanoparticles in the upper gastrointestinal tract after oral administration, an outer coating of sodium alginate and chitosan is continued to be added, and during three cyclic coatings, the surface Zeta potential of the nanoparticles changes. FIG. 4C shows that the absolute value of the surface Zeta potential increases gradually at the same time. The surface Zeta potential of the Chi/Alg-MSN-HA after final coating reaches about −34.13±1.35 mV, which is favorable for dispersion stability in a solvent system.

### 2. Fourier Infrared Spectroscopy Characterization Results and Analysis of a Nano Delivery System

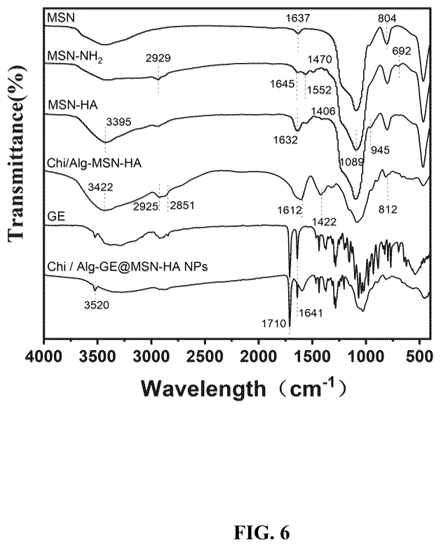

Modifications and functional groups of nanocarriers are confirmed by Fourier Transform Infrared (FTIR) Spectroscopy. FIG. 6 shows FTIR spectrum of MSNs, MSN@NH₂, MSN-HA, Chi/Alg-MSN-HA, Chi/Alg-GE@MSN-HA NPs, and free GE according to some embodiments of the present disclosure. The FTIR spectrum of the MSN@NH₂, the MSN-HA, the Chi/Alg-MSN-HA, the Chi/Alg-GE@MSN-HANPs, and the GE are compared in FIG. 6. The absorption peaks at 1089 cm⁻¹ and 804 cm⁻¹ are attributed to backbone vibrational peaks of Si—O—Si, and a short peak at 804 cm⁻¹ is Si—OH, and all of the above are characteristic peaks of the MSNs. The absorption peak at 2929 cm⁻¹ is inferred to be C—H stretching of a propylamide group. The absorption peaks at 1645 cm⁻¹, 1470 cm⁻¹ are attributed to N—H bending vibration of an amino group. The MSN@NH₂ has a band at 1552 cm⁻¹ that disappears after Chi functionalization and is classified as N—H bending (ON—H). The above peak information proves that the amino group has been modified to the MSNs, and the MSN@NH₂ is successfully synthesized. The strong peak at around 3395 cm⁻¹ is attributed to a hydroxyl absorption peak of hyaluronic acid. The absorption peak at 945 cm⁻¹ is attributed to the antisymmetric out-of-plane vibration of a sugar ring of the hyaluronic acid. The absorption peaks at 1632 cm⁻¹ and 1406 cm⁻¹ are attributed to antisymmetric and symmetric stretching vibrational peaks of —COO— in the hyaluronic acid. The above peak information confirms functionalization of the hyaluronic acid. The large broad peak at 3422 cm⁻¹ is attributed to a large number of hydroxyl groups on the surface of sodium alginate and hyaluronic acid grafted nanoparticles. The absorption peaks at 2925 cm⁻¹ and 2851 cm⁻¹ are attributed to vibrational absorption peaks of methylene groups in the sodium alginate. The absorption peak at 1612 cm⁻¹ may be the C═O vibration of carboxylic acid —COO— in the sodium alginate. The absorption peak at 1422 cm⁻¹ is the vibrational absorption peak of a methyl group in chitosan. The above peak information confirms a successful modification of sodium alginate/chitosan shell coat to the surfaces of nanoparticles. The sharp peak at around 3520 cm⁻¹ is attributed to the stretching vibration of the hydrogen bond-O—H in geniposide. The strong peak at about 1710 cm⁻¹ is attributed to the stretching vibration of an ester carbonyl group in the geniposide. The peak at about 1641 cm⁻¹ is attributed to the stretching vibration of —C═C— in the geniposide. At the same time, characteristic peaks of carriers are affected by interaction with the geniposide, and peak positions are slightly shifted. The above peak information confirms that the geniposide is successfully loaded into the nanoparticles.

### 3. Establishment of a Method for Determining a Content of the Geniposide

Referring to a method for determining of the geniposide in Chinese Pharmacopoeia, chromatographic conditions use octadecylsilane-bonded silica gel as a filler (CAPCELL PAK C18 MGII, a column with 4.6 mm×100 mm); water as mobile phase A, acetonitrile as mobile phase B, water and acetonitrile (in a volume ratio of 15:85) as mobile phase; a flow rate of 0.6 mL per minute; a detection wavelength of 238 nm; and an injection volume of 10 μL. The number of theoretical plates is not less than 1500, calculated with reference to the geniposide peak. Referring to the Chinese Pharmacopoeia for methodological investigations, the investigations include linearity investigation, precision, stability, reproducibility, and spiked recovery.

### Embodiment 1

Embodiment 1 provides a method for preparing the oral targeted nano delivery system containing geniposide.

### 1. Preparation of MSNs

750 mg of CTAB (2.05 mmol) was dissolved in 360 mL deionized water with 400 rpm magnetic stirring (a rotor used is a 50 mm (diameter)*17 mm A-type (olive-type) stirrer) for 20 min at room temperature until completely dissolved to obtain a solution, and 0.21 g of sodium hydroxide was added to the solution. When the CTAB was completely dissolved, an appropriate amount of TMB was quickly added and stirred continuously, and the rotational speed was adjusted to 500 rpm, and the temperature was ramped up to the target temperature of 80° C., and then continued to be heated and stirred magnetically for 2 h.

Then 3.75 mL of TEOS was added dropwise at 1 drop/5 s at 900 rpm using a pressure-equalizing dropping funnel and reacted at 80° C. with a stirring speed of 500 rpm for 2 h. Centrifugation was performed using a low-temperature high-speed centrifuge at 12,000 rpm at 4° C., supernatant was discarded, deionized water was added, vortexed at 100 rpm for 5 min, and centrifugation was performed again at 12,000 rpm for 15 min, and the supernatant was discarded. Washing was repeated three times with deionized water and methanol, respectively, to remove residual reactive substances in a reaction solution, and the obtained lower precipitate was the MSNs. The MSNs were vacuum dried at 60° C. for 24 h under reduced pressure. The MSNs were baked in a muffle furnace at 550° C. for 6 h and then cooled down to room temperature naturally, then resuspended with deionized water and washed by vortexing at 1000 rpm for 5 min, and supernatant containing impurities was discarded by centrifugation at 12000 rpm for 10 min, which was repeated three times, to obtain the MSNs.

### 2. Preparation of MSN@NH₂

The MSNs were dispersed in methanol to obtain a mixture including the MSNs and the methanol, and then, 3-aminopropyltriethoxysilane (APTES) was added to the mixture (MSNs/methanol/APTES=1 g:1 mL:10 μL), and a reaction was carried out at room temperature with magnetic stirring at 300 rpm overnight. The MSN@NH₂ obtained was centrifuged at 12000 rpm for 10 min, washed with methanol, and vacuum-dried under reduced pressure at 60° C. overnight.

### 4. Preparation of MSN-HA

Solution A was obtained by adding 50 mg MSN@NH₂ to a MES buffer solution (adjusted to pH=5.5) and stirring at 400 rpm for 2 h at room temperature. Solution B was obtained by adding 30 mg hyaluronic acid to 6 mL the MES buffer solution (adjusted to pH=5.5) and stirring at 300 rpm for 1 h at room temperature to fully dissolved, and adding 144 mg of EDCI and 86.4 mg of NHS to activate the carboxyl group of the hyaluronic acid and stirring for 1 h at room temperature. The solution A and the solution B were mixed at a volume ratio of 1:1 and stirred at 300 rpm for 24 h at room temperature to obtain the MSN-HA. The MSN-HA was washed three times with deionized water, freeze-dried in a lyophilizer for 48 h, and collected and stored at −20° C.

### 5. Preparation of Chi/Alg@MSN-HA NPs

Nanoparticles were obtained by dispersing 100 mg MSN-HA in 20 ml of 2 mg/ml chitosan solution (pH=6, a phosphate buffered saline (PBS) buffer solution), stirring at 500 rpm for 30 min at room temperature, and then washing three times with PBS buffer solution. The nanoparticles were suspended in 25 ml of 2 mg/ml sodium alginate solution, stirred at 500 rpm for 30 min at room temperature, centrifuged (11,000 rpm, 4° C., 15 min), washed with PBS buffer solution for 3 times, continued to be coated repeatedly and alternately for 3 times according to the above steps, and freeze-dried in a lyophilizer for 48 h to obtain the Chi/Alg@MSN-HA NPs and the Chi/Alg@MSN-HA NPs are stored at −20° C.

### 6. Chi/Alg-GE@MSN-HANPs

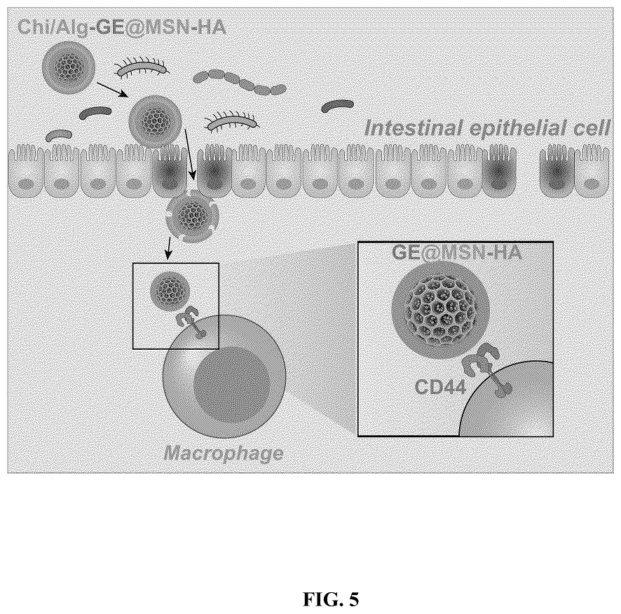

Geniposide and nanocarriers Chi/Alg@MSN-HA NPs in a proportional ratio were added and dispersed in anhydrous ethanol, ultrasonic for 10 min, magnetically stirred at 400 rpm overnight at room temperature, centrifuged at 12,000 rpm for 10 min, and washed with deionized water and anhydrous ethanol repeatedly for 3 times, and geniposide-containing Chi/Alg-GE@MSN-HA NPs were obtained by freeze-drying for 48 h. A schematic diagram is shown in FIG. 5, and an FTIR spectrum is shown in FIG. 6. FIG. 5 is a schematic diagram of Chi/Alg-GE@MSN-HA NPs according to some embodiments of the present disclosure. FIG. 6 shows FTIR spectrum of MSNs, MSN@NH₂, MSN-HA, Chi/Alg-MSN-HA, Chi/Alg-GE@MSN-HA NPs, and free GE according to some embodiments of the present disclosure.

The same method was carried out to load geniposide with nanocarriers Chi/Alg-MSN-HA in a proportional ratio of W(GE)/W(Chi/Alg-MSN-HA) of 1:10, 1:8, 1:4, 1:2, 1:1, 2:1, 4:1, 10:1, respectively. As shown in Table 4, an optimization was carried out using a drug loading efficiency and a drug releasing rate as indicators, and an optimum drug loading ratio, i.e., W(GE)/W(Chi/Alg-MSN-HA), was screened as 1:1, and all subsequent experiments were carried out in the proportional ratio of the W(GE)/W(Chi/Alg-MSN-HA of 1:1.

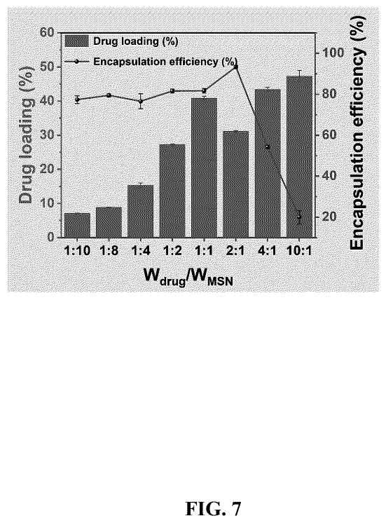

Results of a plurality of test data under several optimized conditions show that a particle size of nano drug was not obvious changed with increase of dosage of the geniposide when the W(GE)/W(Chi/Alg-MSN-HA) was in a range of 1:10 to 1:2. The insignificant change in the particle size indicates that main form of drug loading of Chi/Alg-MSN-HA may be through van der Waals force of an internal structure of mesopores and an adsorption force of an internal cavity, and silica hydroxyl group contained in the MSNs and hydroxyl group in the geniposide may form hydrogen bond to enhance an effect force of cavity sites inside the mesoporous on the geniposide, inferring that most of the drug mainly existing inside cavities of carriers. When the W(GE)/W(Chi/Alg-MSN-HA) was in a range of 1:2 to 10:1, the particle size gradually increased with the W(GE)/W(Chi/Alg-MSN-HA) continued to increase. Significant increase in the particle size indicates that surface adsorption of the carriers causes the drug to bind to the outer surface of the particles, which may depend on the van der Waals force to attract each other, causing the drug molecules to adsorb on the pore wall and surface of the MSNs. Electrostatic interactions due to the nature of charge on the surface of the carriers and residual silica hydroxyl groups may play a major role, and at this stage, the drug is gradually attached to the surface of the nanoparticles, thereby affecting the particle size of the nanodrug. FIG. 7 shows drug loading efficiency and encapsulation efficiency test results (n=3) of Chi/Alg-GE@MSN-HA with different feeding ratios according to Embodiment 1 of the present disclosure. Curves of the drug loading efficiency and the encapsulation efficiency in FIG. 7 shows that when the drug loading ratio is in a range of 1:10 to 1:1, the drug loading efficiency increases, and the encapsulation efficiency does not float much with the increase of W(GE)/W(Chi/Alg-MSN-HA). The drug loading efficiency is basically stable when a drug loading ratio is in a range of 2:1 to 10:1, while the encapsulation efficiency is gradually decreased, which indicates that drug loading capacity of carriers is basically saturated when the drug loading ratio is 1:1. Results of Zeta potential show that the Zeta potential does not change significantly with the drug loading ratio in a range of 1:10 to 1:1, while the Zeta potential increases when the drug loading ratio is in a range of 1:1 to 10:1, which may be related to a fact that the increase in the drug loading capacity of geniposide with weak acidity to a certain extent would affect a surface electron cloud density of nanocarriers (FIG. 7). According to the guideline for 9014 particulate formulations of Chinese Pharmacopoeia 2020 Edition, the encapsulation efficiency should generally not be lower than 80%. Therefore, according to a requirement of the encapsulation efficiency, only W(GE)/W(Chi/Alg-MSN-HA) of 1:2, 1:1, and 2:1 among different drug loading ratios fulfills the requirement and only W(GE)/W(Chi/Alg-MSN-HA) of 1:1 shows the highest drug loading capacity of 40.84%+0.53% (as shown in Table 4). Therefore, combining the above four indexes of the particle size, the Zeta potential, the drug loading efficiency, and the encapsulation efficiency, the best drug loading ratio W(GE)/W(Chi/Alg-MSN-HA) of 1:1 was used to prepare the nanomedicine Chi/Alg-GE@MSN-HA for the subsequent studies.

TABLE 4

| W(GE)/W(Chi/Alg- | | Zeta | Drug loading | Encapsulation |
|---|---|---|---|---|
| MSN-HA) | Particle size/nm | potential/mv | efficiency (%) | effciency (%) |
| 1:10 | 128.24 ± 3.72 | −32.15 ± 1.85 | 7.04 ± 0.17 | 77.39 ± 1.88 |
| 1:8 | 129.35 ± 2.21 | −31.78 ± 2.05 | 8.83 ± 0.09 | 79.47 ± 0.77 |
| 1:4 | 126.18 ± 2.24 | −34.10 ± 2.41 | 15.31 ± 0.73 | 76.57 ± 3.67 |
| 1:2 | 130.06 ± 3.66 | −30.15 ± 3.01 | 27.19 ± 0.32 | 81.58 ± 0.96 |
| 1:1 | 138.61 ± 2.15 | −30.15 ± 2.88 | 40.84 ± 0.53 | 81.68 ± 1.06 |
| 2:1 | 140.15 ± 3.11 | −35.16 ± 2.97 | 31.12 ± 0.27 | 93.35 ± 0.81 |
| 4:1 | 142.77 ± 6.10 | −36.11 ± 2.71 | 43.41 ± 0.63 | 54.26 ± 0.79 |
| 10:1 | 142.05 ± 3.07 | −36.08 ± 2.11 | 47.25 ± 1.74 | 19.96 ± 3.19 |

### Embodiment 2

### Evaluation of the Drug Loading Efficiency, the Encapsulation Efficiency, and Stability of Chi/Alg@MSN-HA NPs

Embodiment 2 assesses the stability of the Chi/Alg@MSN-HA NPs after long-term frozen storage. The dried Chi/Alg-GE@MSN-HA NPs were placed at a low temperature of 4° C., and after 0, 7, 14, 21, 30, 45, and 60 days, a small amount of the Chi/Alg-GE@MSN-HA NPs were re-dispersed into deionized water, and the particle size, the PDI, and the Zeta potential were measured, and measurement results were recorded on days 0, 7, 14, 21, 30, 45, and 60 days, respectively. The stability of the Chi/Alg@MSN-HA NPs was analyzed based on the measurement results after long-term frozen storage.

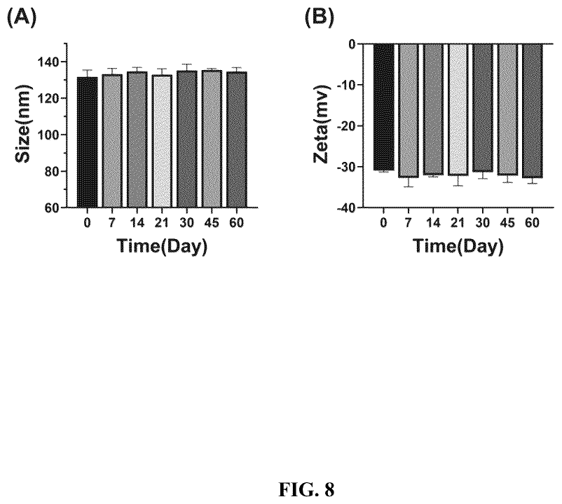

Changes in physicochemical properties of the Chi/Alg-GE@MSN-HA NPs in the case of long-term frozen storage were examined, and the particle size, the Zeta potential, and dispersibility were selected as indexes for evaluating the stability of the Chi/Alg-GE@MSN-HA NPs in Embodiment 2. FIG. 8 shows changes (n=6) in particle size and Zeta potential of Chi/Alg-GE@MSN-HA NPs sampled on different days during long-term frozen storage according to Embodiment 2 of the present disclosure. As shown in FIG. 8, with prolongation of the long-term frozen storage, the particle size and Zeta potential of the Chi/Alg-GE@MSN-HA NPs were examined by selecting different days for sampling over a period of 60 days. After redispersed in deionized water, the particle size is stabilized at a range of 130 nm to 135 nm, and the PDI is less than 0.3, indicating good stability. A carrier skeleton and the particle size of the Chi/Alg-GE@MSN-HA NPs have not changed significantly due to a long-term storage, and the mesopores and the outer shell coat have not changed significantly, which can be speculated that the drug loading capacity and drug releasing behavior of the Chi/Alg-GE@MSN-HA NPs should be preserved to a great extent, and the drug encapsulated inside the carrier and protected by the coating layer is favorable for avoiding decomposition or oxidative deterioration under long-term storage due to poor drug stability. At the same time, an absolute value of surface charge Zeta potential is stabilized above-30 mV for a long period, which indicates that the surface charge nature was preserved to a great extent, and therefore performance of nanocarriers was well maintained in the long-term storage study.

2. Evaluation of Drug Release from Nanocarriers

Release profiles of the Chi/Alg-GE@MSN-HA NPs and free GE were determined in vitro using the dialysis method. 3 mL of the Chi/Alg-GE@MSN-HA NPs and the free GE were added to a dialysis bag, respectively. The dialysis bag was suspended in simulated gastric fluid (sgf) (pH=1.2, 40 mL, 1% Tween 80) and stirred at 100 rpm for 2 h at 37° C. to simulate intragastric conditions. The dialysis bag was then suspended in simulated intestinal fluid (sif) (pH=6.8, 40 mL, 1% Tween 80) stirred at 100 rpm at 37° C. for 4 hours to simulate conditions in the small intestine. Finally, the dialysis bag was further incubated in simulated human colonic fluid (scf) (pH=7.4, 40 mL, 1% Tween 80) stirred at 100 rpm at 37° C. for 8 hours. At a preset time point, 1 mL of new dissolution medium was added after removing 1 mL of sample. Some measurement errors mainly due to short durations in the sgf, the sif, and the scf were overcome. A dialysis bag containing the Chi/Alg-GE@MSN-NH₂ NPs and a dialysis bag containing the free GE were placed into the sgf at the same time, and a concentration of the drug in the filtrate was measured by high-performance liquid chromatography (HPLC) at a preset time point. The sif and the scf are operated in the same manner as the sgf, and an operation of the sgf is described above.

The drug loading efficiency (%)=a mass of the geniposide in nanocarriers/a total mass of nanodrugs×100%;

The encapsulation efficiency (%)=a mass of the geniposide in the nanocarriers/a total feeding mass of geniposide×100%;

### 2.1 an Examination Result and Analysis of a Linear Relationship of a Method for Detecting the Geniposide

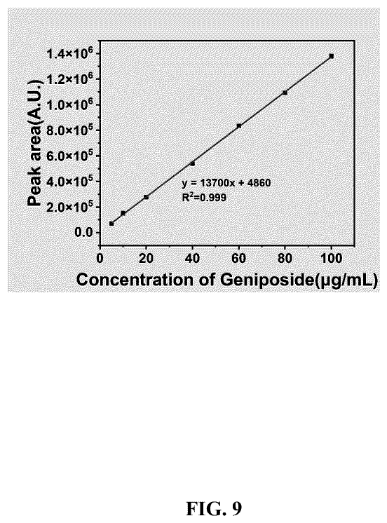

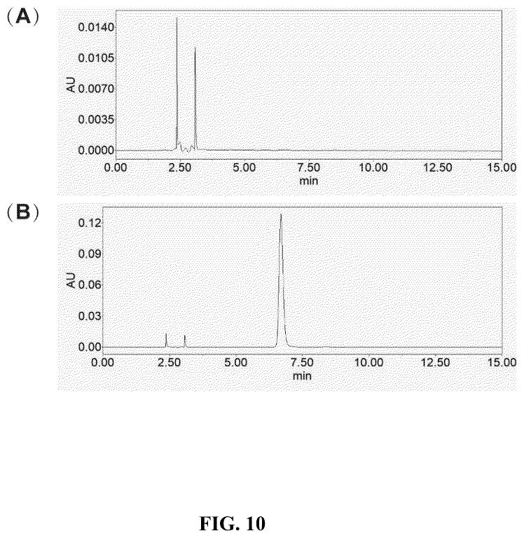

FIG. 9 shows a concentration standard curve of geniposide solution according to Embodiment 2 of the present disclosure. The examination result of the linear relationship is shown in FIG. 9, and values of peak areas of response chromatograms for the geniposide with a concentration range of 5~100 μg/mL are recorded. The concentration standard curve was plotted with the concentration (μg/mL) as a horizontal coordinate and the peak area as a vertical coordinate, and a linear regression equation was obtained as: y=13700x+4860 (R²=0.999), which indicated that the linear relationship was good in the concentration range of 5~100 μg/mL. FIG. 10 shows HPLC chromatogram profiles according to Embodiment 2 of the present disclosure, where (A)—blank solvent, and (B)—geniposide standard solution. The HPLC chromatogram of the geniposide standard solution is shown in FIG. 10, with the chromatographic peak exhibiting a retention time of 7.101 min.

### 2.2 Results and Analysis for In Vitro Drug Release

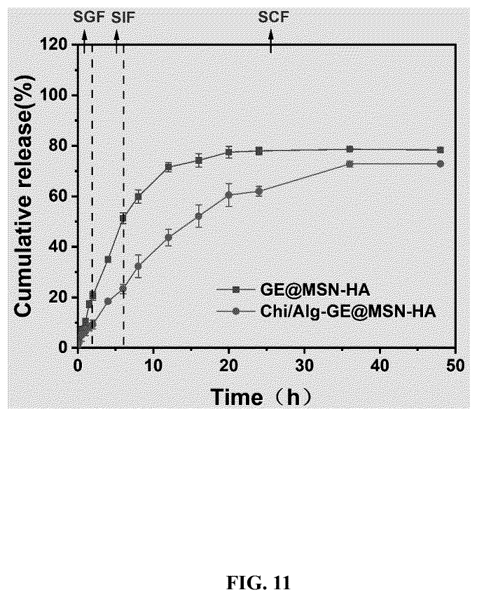

Different parts of the gastrointestinal tract in the body have different pH values. Usually, the stomach is strongly acidic (typically pH 1.5-3.5), the small intestine is weakly alkaline (approximately pH 6-7.5), and the colon has a relatively high pH (generally between 7 and 8). The Chi/Alg-GE@MSN-HA NPs prepared the example primarily utilize pH-responsive mechanisms to activate drug release. By investigating the drug-releasing behavior of the Chi/Alg-GE@MSN-HA NPs, in vitro simulation of the fluid environment of the stomach, the small intestine, and the colon (0-2 h in the sgf, 2-6 h in the sif, and 6-48 h in the scf) was used to determine release profiles of GE in GE@MSN-HA NPs and the Chi/Alg-GE@MSN-HA NPs. FIG. 11 shows cumulative release profiles (n=6) of GE@MSN-HA NPs and Chi/Alg-GE@MSN-HA NPs under a simulated gastrointestinal condition in vitro according to Embodiment 2 of the present disclosure. The result shows that Cumulative GE release rates of the GE@MSN-HA NPs and the Chi/Alg-GE@MSN-HA NPs in the sgf, in 0 to 2 h, at 2 h reach 20.58±1.74% and 9.20±1.73%, respectively. Cumulative GE release rates of the GE@MSN-HA NPs and the Chi/Alg-GE@MSN-HA NPs in the sif, in 2 h to 6 h, at 6 h reach 51.30±2.18% and 23.32±1.88%, respectively. Cumulative GE release rates of the GE@MSN-HA NPs and the Chi/Alg-GE@MSN-HA NPs in the scf, in 6 h to 48 h, at 48 h reach 78.32±0.69% and 72.76±0.65%, respectively. The cumulative release profiles show that the GE@MSN-HANPs were almost completely released at 24 h with a cumulative release rate of 77.90±1.49%, while the Chi/Alg-GE@MSN-HA NPs were almost completely released at 48 h (FIG. 11). Due to different biodegradation properties of outer polymers, swelling and degradation behaviors of the sodium alginate and chitosan hydrogels, in particular, are sensitive to pH. The GE@MSN-HA NPs are intolerant to an acidic condition of the simulated environment of the stomach due to the lack of outer coating of the sodium alginate and the chitosan, and thus are rapidly released in the simulated environments of the stomach and the small intestine, and prematurely reach a saturation point of cumulative release. In the simulated environments of the stomach and the small intestine, the Chi/Alg-GE@MSN-HA NPs are relatively stable and tolerant to ambient acidity due to a relatively stable structure of the outer modified coating. The drug is released slowly, and only in response to reaching the colon, where the simulated environmental conditions led to the disintegration of the outer shell layer to release inner materials, and a layer of the hyaluronic acid is further disintegrated to release the GE. Therefore, the in vitro simulation of the release results shows that the Chi/Alg-GE@MSN-HA NPs have a certain slowing effect on the GE release behavior.

### 2.3 In Vivo Evaluation of an Oral Targeted Delivery System Containing Geniposide for Anti-UC

2.3.1 an Oral Targeted Delivery System without the Geniposide

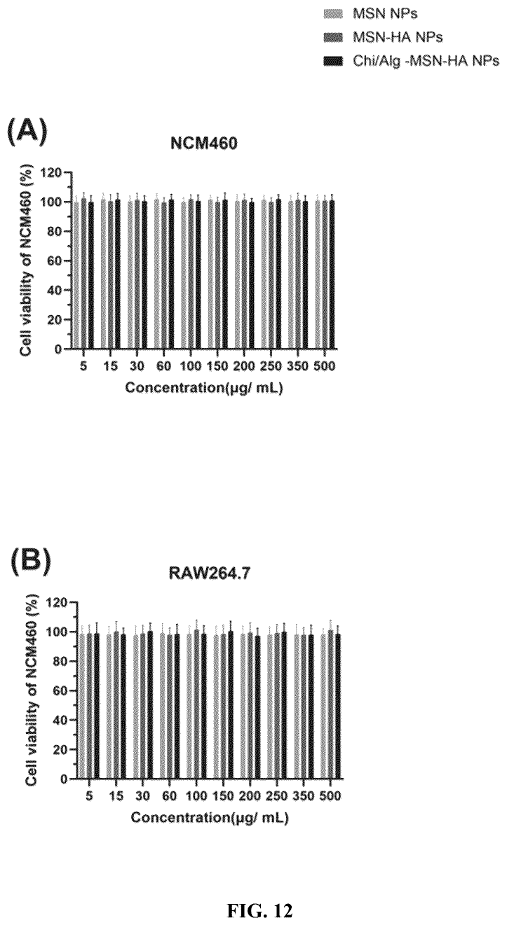

\textcircled{1} Test the nanocarriers for cytotoxicity. Two cell lines, NCM460 cells and RAW264.7 cells, were selected. Administration groups included MSNs, MSN-HA NPs, and Chi/Alg-MSN-HA NPs. Safety was assessed at a cellular level using a cell counting kit-8 (CCK-8) method. FIG. 12 is a diagram illustrating cytotoxicity evaluation (n=6) of MSNs, MSN-HA NPs, and Chi/Alg-MSN-HA NPs according to Embodiment 2 of the present disclosure. Cell viability has been detected by the CCK-8 method and combined with observation of cell morphology, and the results have shown that when the MSNs, the MSN-HA NPs and the Chi/Alg-MSN-HA NPs are in a concentration range of 5-500 μg/ml, more than 90% cell viability was maintained and had intact cell morphology, and any obvious apoptosis has not been observed (FIG. 12). Comprehensively, it is shown that the MSNs have not exhibit significant cytotoxicity against the NCM460 cells and the RAW264.7 cells in a concentration range below 500 μg/ml. The result provides important safety data support for potential application of the Chi/Alg-MSN-HA NPs in drug carriers, bio-imaging, and also provides an important basis for further study of targeting performances and drug efficacy evaluation after drug delivery.

\textcircled{2} Investigate the targeting performances of the nanocarriers in a lipopolysaccharide (LPS)-induced RAW264.7 inflammation model, confocal fluorescence images were used to analyze uptake and distribution of free coumarin 6 (C6), C6@MSN-HA NPs, and Chi/Alg-C6@MSN-HA NPs after incubation for 30 min, 1 h, 2 h, and 4 h.

According to the existing literature, expression of CD44 receptor in the RAW264.7 cells after LPS-induced is significantly increased, which is favorable for the specific targeting and tracking of the nanoparticles C6@MSN-HANPs of hyaluronic acid-modified for the RAW264.7 cells.

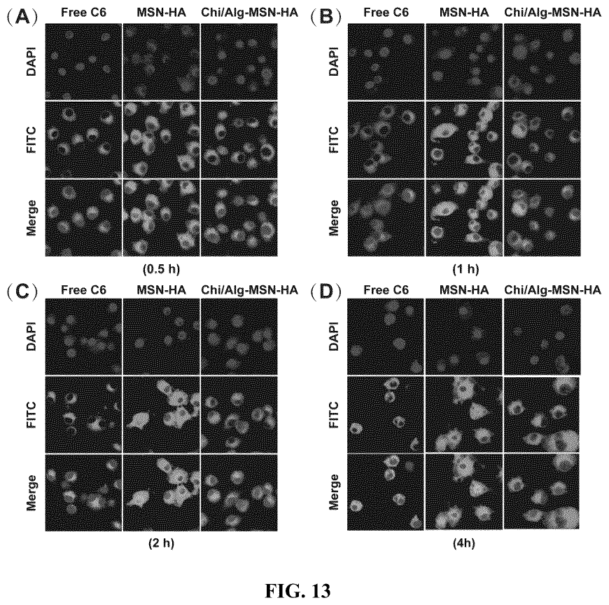

FIG. 13 shows CLSM analysis of uptake and distribution of LPS-induced RAW264.7 inflammation model after 30 min, 1 h, 2 h, and 4 h incubation with free C6, C6@MSN-HA NPs, and Chi/Alg-C6@MSN-HA NPs in vitro (n=6) according to Embodiment 2 of the present disclosure. It can be seen from a CLSM image in FIG. 13 that, fluorescence of free C6 is weak in the cells and is present mainly at the periphery of the cells. From the fluorescence display in the cells, the RAW264.7 cells rarely uptook the Chi/Alg-C6@MSN-HA NPs in 4 h. For the C6@MSN-HA NPs, the RAW264.7 cells show a significant increase in C6 uptake, and the intensity of the uptake is time-dependent during 4 h. The accumulation of the C6@MSN-HANPs in the RAW264.7 cells is significantly higher than that of the Chi/Alg-C6@MSN-HA NPs (p<0.05) by incubating up to 4 h, which may be related to the stability of the outer sodium alginate/chitosan shell coat layer in the culture environment. In contrast, HA on surface exposed to the cell environment due to lack of protection of the sodium alginate/chitosan (Chi/Alg) shell coat layer in the outer layer of the C6@MSN-HA NPs may directly bind to the CD44 receptor on the macrophage surface, thereby enhancing the targeting performances of the C6@MSN-HA NPs. The results indicate that the C6@MSN-HA NPs may actively target the CD44 receptor and enter the RAW264.7 cells through endocytosis. A plurality of inflammation-activated macrophages accompanied the colonic lesions of UC patients, so the ability of the GE@MSN-HA NPs to target the macrophages provides an effective way for the inflammation-site targeted delivery of GE to exert therapeutic effects. Meanwhile, the protective effect of the Chi/Alg shell coat layer can enable the Chi/Alg-C6@MSN-HA NPs to avoid destruction in the upper gastrointestinal tract to reach the colon, and combined with targeting macrophage performance of the MSN-HA NPs, to achieve localized drug release in the lesion.

(iii) Investigate the effect of the Chi/Alg-GE@MSN-HA NPs on targeting colon lesions in UC murine model. Using a small animal in vivo imaging technique, a 1,1′-dioctadecyl-3,3,3′,3′-tetramethylindotricarbocyanine iodide (Dir) fluorescent probe was used to replace the drug to be loaded in the nanodelivery system to obtain Chi/Alg-Dir@MSN-HA NPs, and a dynamic process of in vivo delivery of nanoparticles was observed and analyzed.

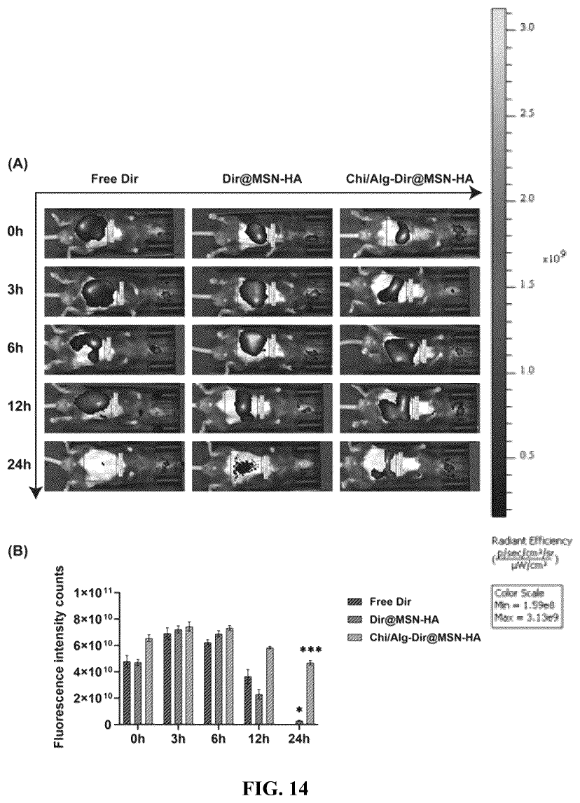

FIG. 14 shows results of colon-targeting verification of an oral nanocarrier system Chi/Alg-Dir@MSN-HA in mice according to Embodiment 2 of the present disclosure, where (A)—in vivo imaging photographs of the mice administered free Dir, Dir@MSN-HA, and Chi/Alg-Dir@MSN-HA by gavage in 0-24 h, and (B)—in vivo fluorescence intensity statistics at different time points (n=6), (compared to a free Dir group, *p<0.05 and ***p<0.001). As shown in FIG. 14, a free Dir fluorescent probe peaked around 3 h after entering the body, and then was rapidly excreted afterward, showing no retention property in the intestine. Compared to the free Dir fluorescent probe, the Dir@MSN-HA NPs were maintained in vivo at peak fluorescence intensity for 6 h before rapid excretion. Possibly, the protective effect of the nanoparticles on the Dir probes slowed down the early release of the Dir fluorescent probes, while a layer of the hyaluronic acid on the surface, although destroyed in the upper gastrointestinal tract, provided a certain degree of protection on the outside of the particle, delaying the exposure of the Dir to adverse conditions in the in vivo environment, and there was still a little fluorescence in the UC murine model at 24 h. The Chi/Alg-Dir@MSN-HA NPs show strong retention in in vivo imaging after fluorescence peaked at 3 h, fluorescence intensity at 24 h is still high, which is significantly different (p<0.001) compared to the free Dir group. The above results indicate that the Chi/Alg-Dir@MSN-HA NPs may achieve targeting at the colonic inflammatory site with strong retention, which is favorable for the localized treatment of the drug GE at the colonic inflammatory site, thereby improving the intrabody targeting and safety of the drug.

\textcircled{4} Observe whether the Chi/Alg-Dir@MSN-HA achieves targeted accumulation in the UC murine model by administering the free Dir, the Dir@MSN-HA, and the Chi/Alg-Dir@ MSN-HA by gavage to the UC murine model and in vitro imaging of the colon tissue isolated after 24 h.

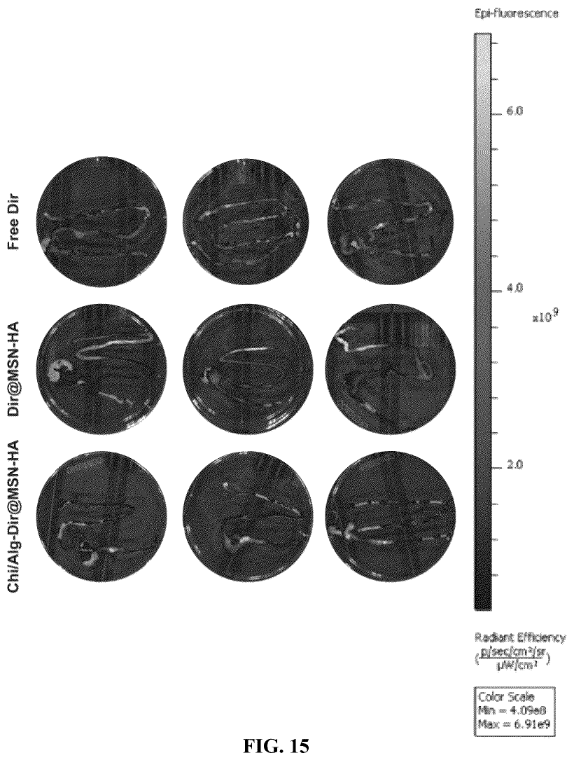

FIG. 15 shows in vitro imaging photographs of colon isolated from mice after gavage administration of free Dir, Dir@MSN-HA, and Chi/Alg-Dir@MSN-HA for 24 h according to Embodiment 2 of the present disclosure (n=3). Results as shown in FIG. 15 indicate that the Dir fluorescence in the mice administered free Dir group is widely distributed in the intestines and does not have site-specific accumulation. The relatively stronger fluorescence intensity of the Dir@MSN-HA group at the small intestine site, compared to the free Dir group, may be related to the lack of protection of the sodium alginate/chitosan (Chi/Alg) shell coat layer in the outer layer of the Dir@MSN-HA particles, which allows the Dir@MSN-HA particles to disintegrate earlier in the upper gastrointestinal tract to release the probe, and combined with the outer layer of nanoparticles to provide a certain degree of protection, thereby delaying the in vivo excretion of the Dir. Compared to the free Dir group, 24 h after entering the UC murine model, the Chi/Alg-Dir@MSN-HA NPs carrying the Dir probes shows higher fluorescence intensity at the colon site, which is significantly stronger than that at other sites in the intestine, indicating the targeted accumulation of the Chi/Alg-Dir@MSN-HA in the inflammatory site of the colon in the UC murine model, which provides a better therapeutic manner for delivering the GE for the treatment of UC symptoms, and is favorable for the improvement of targeting, efficacy, and safety of the GE for the UC.

### 2.3.2 In Vivo Pharmacodynamic Evaluation of the Chi/Alg-GE@MSN-HA

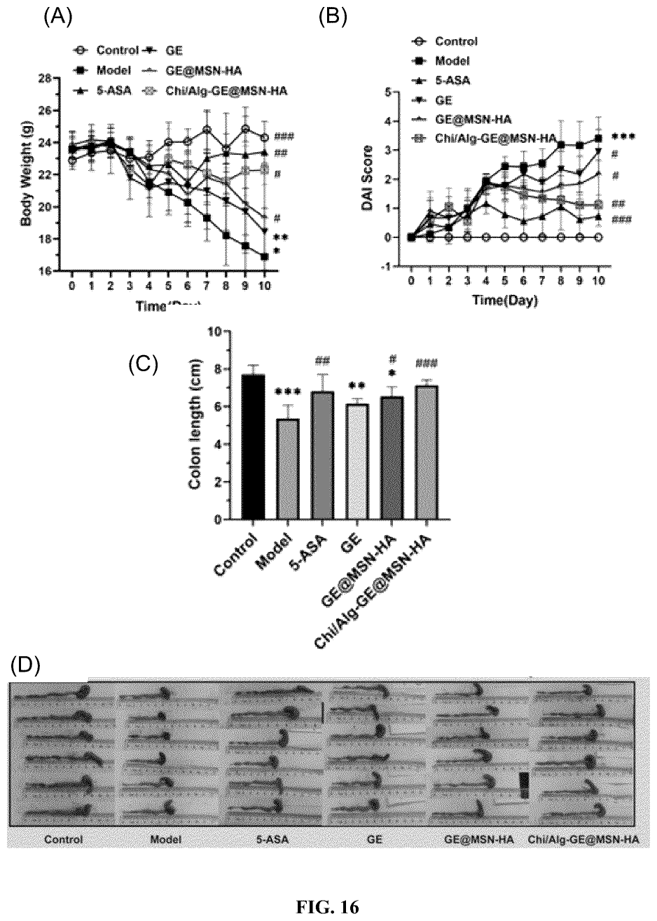

Verification of the efficacy of the Chi/Alg-GE@MSN-HA in targeted delivery of the GE for the UC. The therapeutic efficacy was comprehensively evaluated by monitoring general signs, behavioral conditions, changes in body weight, disease activity index (DAI) scores, and a colonic length in the UC murine model. Observation of the general signs revealed that the mice in the model group had insignificant changes in the indexes in the first three days of the modeling, and then experienced a sudden drop in the body weight and appearance of blood in the stools one after another on the fourth and fifth days, and most of the mice were curled up, motionless at touch, and in a weak state after the eighth day. Compared to the model group, the administered group improved the above conditions, especially by the end of the experiment, the mice in the Chi/Alg-GE@MSN-HA group were in good condition, with normal locomotor responses and normal defecation, and no blood was obvious in the stool. FIG. 16 shows in vivo efficacy evaluation of Chi/Alg-GE@MSN-HA according to Embodiment 2 of the present disclosure; where (A)—change curves in body weight of the mice during an administration observation period; (B)—DAI scores of the mice; (C)—statistics of colonic lengths of a plurality of groups of mice; and (D)—photographs of the colons (n=6); (compared to the control group, *p<0.05, **p<0.01, and ***p<0.001; compared to the model group, *p<0.05, ##p<0.01, and ###p<0.001). Results of the changes in body weight of the mice in each group during the observation period are shown in FIG. 16A. The mice in the model group have lost an average of 6.1 g in body weight, which has seriously affected the living conditions of the mice. Mice in the positive drug 5-ASA group show significant improvement in body weight (compared to the control group, p<0.001). There is a slight improvement in the GE group and the GE@MSN-HA group by gavage. The Chi/Alg-GE@MSN-HA has great effect for weight loss recovery in the UC murine model (compared to the model group, p<0.001), which indicates that the release of the GE by targeting the colon by the Chi/Alg-GE@MSN-HA is more favorable for the development of anti-UC efficacy than that by gavage of the GE, and significantly alleviate the loss of the body weight of the UC murine model, which is associated with the improvement of the drug in localized distribution of the colonic lesions. The DAI scores are used to assess the severity of the UC murine model, mainly from a percentage of body weight loss, stool consistency, and blood in stool. Results of the DAI scores are shown in FIG. 16B. By the end of the experiment, the average DAI score of the mice in the model group is 3.40, which indicates that symptoms of blood in stools, the body weight loss, and defecation were more severe. The Chi/Alg-GE@MSN-HA group shows significant improvement in the DAI scores compared to the GE group by gavage and the GE@MSN-HA group, with an average DAI score of 1.11 (compared to the model group, p<0.01). In addition, the results of the colonic lengths are shown in FIG. 16 C-D, average values of the colonic lengths of the mice in the control group, the model group, the 5-ASA group, the GE group by gavage, the GE@MSN-HA group, and the Chi/Alg-GE@MSN-HA group are 7.72 cm, 5.36 cm, 6.83 cm, 6.16 cm, 6.55 cm, 7.13 cm, respectively. The Chi/Alg-GE@MSN-HA group significantly improves colon shortening symptoms compared to the GE group and the GE@MSN-HA group. Combined with prior in vivo imaging localization results, these findings collectively indicate that the outer shell coat layer of the Chi/Alg-GE@MSN-HA NPs effectively protects GE for targeted release at UC lesions, improving anti-UC efficacy of the GE.

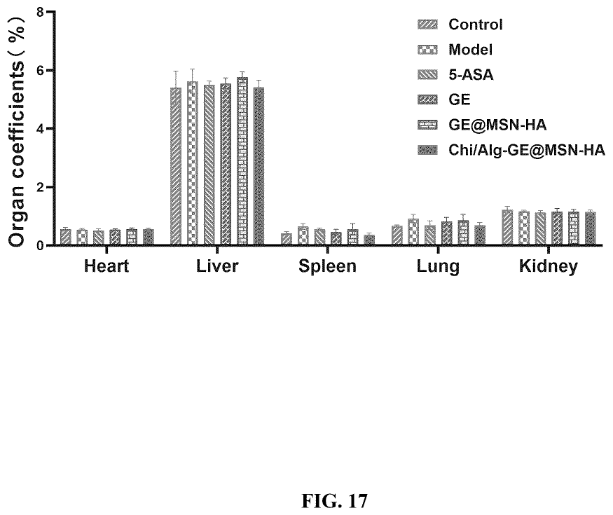

Meanwhile, investigate in vivo safety of the Chi/Alg-GE@MSN-HA in the UC murine model, organ coefficients of major organs are used as an index for preliminary evaluation of the safety. FIG. 17 shows organ coefficient assessments of UC murine model of a plurality of groups of mice (n=6) after administration according to Embodiment 2 of the present disclosure. Results as shown in FIG. 17 shows that, compared to normal mice in the control group (P>0.05), no significant changes in the index of the major organs (e.g., the liver, the heart, the spleen, the lungs, and the kidney) of mice treated with different drugs in other groups. The results of the preliminary evaluation of the safety indicate that the orally administered colon-targeted nanotherapeutic Chi/Alg-GE@MSN-HA NPs demonstrated no significant in vivo toxicity in the UC murine model.

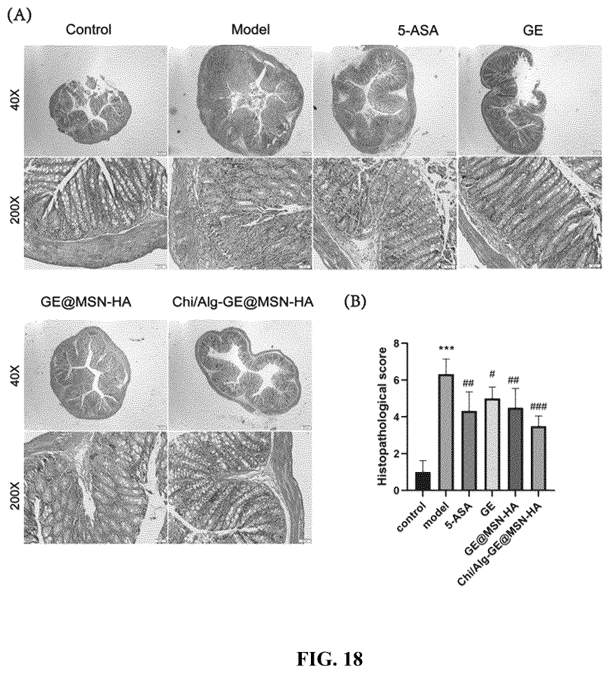

HE staining technique was used to stain the colon of the mice in each group to observe the morphology of the colon. FIG. 18 shows histopathological paraffin sections of mouse colons according to Embodiment 2 of the present disclosure; where, (A)—H&E staining; and (B)—histopathological scores (n=6) (compared to the control group, ***p<0.001; compared to the model group, ^{#}p<0.05, ^{##}p<0.01, and ^{###}p<0.001). As shown in FIG. 18A, the mice in the control group had an intact colon structure. The colons of the mice in the model group show extensive mucosal disruption, with different degrees of inflammatory infiltration in the mucosa and muscularis, and the crypts show irregular damage. Colonic mucosal repair has been observed in the GE group by oral administration, with some recovery of epithelial cells. The Chi/Alg-GE@MSN-HA nanoparticle group exhibited the most significant restoration of colonic mucosa and epithelial cells of the mice. Colonic pathological features in each group have been scored by the indexes of epithelial cell intactness, the degree of inflammatory infiltration, etc., and results are shown in FIG. 18 B. The mice in the Chi/Alg-GE@MSN-HA group have had the best colonic recovery, which is significantly different from the GE group by oral administration (p<0.05).

An optimal drug loading ratio, W(GE)/W(Chi/Alg-MSN-HA), of the oral targeted nano delivery system containing the geniposide in the present disclosure is 1:1, a highest drug loading capacity is 40.84%±0.53%, and the encapsulation efficiency is 81.68±1.06, which complies with the pharmacopoeia requirements.

The oral targeted nano delivery system containing the geniposide in the present disclosure has obvious efficacy, and is safe and non-toxic, which can improve signs of the mice, reduce the DAI scores, and significantly improve UC symptoms, for example, colon shortening, blood in stool, diarrhea, and reduce colon inflammation shown by the HE results. The main organs have not shown obvious toxic pathological changes. The targeting efficiency is high in vitro and in vivo, and the oral targeted nano delivery system containing the geniposide shows good anti-UC efficacy in the in vivo pharmacodynamic evaluation of the DSS-induced UC murine model.

The present disclosure has the following effects.

A modified Stöber method is utilized in the present disclosure to synthesize the MSNs combined with modification of the natural polysaccharide, the hyaluronic acid, and the chitosan/sodium alginate hydrogel coating to prepare the oral drug delivery system that can actively target adhesion at the site of colon inflammation to release the drug and at the same time avoid to degrade in the upper gastrointestinal tract after oral administration.

The oral targeted nano delivery system containing the geniposide can replace traditional enema drug delivery and can largely improve the compliance of the geniposide by oral administration and the targeting of the colon therapy.

The nano delivery system can realize the slow release of the drug, maintain the effective concentration of the drug in the body, and prolong the effective time of the drug, which is of great significance for the treatment of chronic inflammatory diseases (e.g., ulcerative colitis), and better control of the disease progression.

The geniposide is well protected in the nano delivery system, which can avoid degradation and be deactivated in the gastrointestinal tract, improve the stability and bioavailability of the drug, and ensure that the drug can effectively play a therapeutic role.

The present disclosure constructs an oral nano-targeted delivery system containing geniposide to alter the administration site, achieve localized release of geniposide in the colon, enhance therapeutic efficacy against UC, and provide a solution to address safety concerns of geniposide.

While the present disclosure has been described in detail above with a general description and specific embodiments, some modifications or improvements made on the basis of the present disclosure are apparent to one skilled in the art. Therefore, the modifications or improvements made without departing from the spirit of the present disclosure fall within the scope of protection claimed herein.

What is claimed is:

1. A method for preparing an oral targeted nano delivery system containing geniposide, comprising:
step (a): preparation of a targeted nano delivery system
synthesizing mesoporous silica nanoparticles (MSNs) with a Stöber method using a template; removing the template, modifying a surface of the MSNs with amino groups to obtain MSN@NH₂; adding hyaluronic acid (HA) activated by N-hydroxysuccinimide (NHS)/1-ethyl-3-(3-dimethylaminopropyl) carbodiimide hydrochloride (EDCI) to a suspension of the MSN@NH₂, and reacting to obtain MSN-HA; utilizing the MSN-HA, a chitosan solution, and sodium alginate to prepare Chi/Alg@MSN-HA nanoparticles (NPs); and
step (b): preparation of geniposide-loaded Chi/Alg-GE@MSN-HA NPs loading the geniposide with the Chi/Alg@MSN-HA NPs in a proportional ratio to prepare the geniposide-loaded Chi/Alg-GE@MSN-HA NPs.

2. The method according to claim 1, wherein in the step (a), synthesizing the MSNs with the Stöber method includes: using cetyltrimethylammonium bromide (CTAB) as the template, 1,3,5-trimethylbenzene (TMB) as a pore expander, and tetraethyl orthosilicate (TEOS) as a silicon source.

3. The method according to claim 1, wherein in the step (a), modifying the surface of the MSNs with the amino groups includes: dispersing the MSNs in methanol, adding 3-aminopropyltriethoxysilane (APTES), reacting at room temperature overnight, centrifuging an obtained product, washing with methanol, and drying to obtain the MSN@NH₂.

4. The method according to claim 3, wherein a ratio of the MSNs:the methanol:the APTES=0.8-1.2 g:0.8-1.2 mL:0.8-1.2 μL.

5. The method according to claim 1, wherein the step (a) includes dispersing the MSN@NH₂ in a 4-morpholineethanesulfonic acid (MES) buffer solution to obtain the suspension of MSN@NH₂.

6. The method according to claim 1, wherein in the step (a), the preparation of the Chi/Alg@MSN-HA NPs includes dispersing the MSN-HA in the chitosan solution, stirring at room temperature, suspending a resulting product in a sodium alginate solution, stirring to react, and centrifuging to obtain the Chi/Alg@MSN-HA NPs.

7. The method according to claim 1, wherein in the step (b), the preparation of the geniposide-loaded Chi/Alg-GE@MSN-HA NPs includes dispersing the geniposide and the Chi/Alg@MSN-HA NPs in anhydrous ethanol in the proportional ratio, subjecting to ultrasonication, stirring at room temperature overnight, centrifuging, washing repeatedly with deionized water and anhydrous ethanol, and freeze-drying to obtain the geniposide-loaded Chi/Alg-GE@MSN-HA NPs.

8. The method according to claim 1, wherein in the step (b), a mass ratio of the geniposide to the Chi/Alg@MSN-HA NPs is in a range of 1:10 to 10:1.

9. An oral targeted nano delivery system containing geniposide, wherein the oral targeted nano delivery system containing geniposide is prepared by the method of claim 1.
